(19) United States
(12) Patent Application Publication
(10) Pub. No.: US 2011/0224535 A1
(43) Pub. Date: Sep. 15, 2011

(54) MAGNETIC RESONANCE-ASSISTED BIOPSY EXAMINATION METHOD, AND A MAGNETIC RESONANCE SYSTEM FOR IMPLEMENTING THE METHOD

(76) Inventors: Martina Doehlemann, Erlangen (DE); Petra Tamara Giesen, Oberasbach (DE); Marion Hellinger, Uttenreuth (DE)

(21) Appl. No.: 13/044,793

(22) Filed: Mar. 10, 2011

(30) Foreign Application Priority Data
Mar. 10, 2010 (DE) 10 2010 002 723.5

(51) Int. Cl. A61B 5/055; A61B 10/02

(52) U.S. Cl. 600/411

(57) ABSTRACT

In a magnetic resonance-assisted biopsy method for a biopsy examination, using magnetic resonance measurement executed by a magnetic resonance measurement unit and a biopsy unit and a computer console, the computer console is located separately from the magnetic resonance measurement unit, the following steps are implemented:

a preparation step to position a patient on a patient bed,

a measurement step to acquire a magnetic resonance measurement of a region of the patient that is relevant to the biopsy examination,

a planning step to plan a sample extraction,

a biopsy step for a sample extraction from the region of the patient that is relevant to the biopsy examination.

The operator is guided through the preparation step and/or the measurement step and/or the planning step and/or the biopsy step in a computer-controlled manner.

### BACKGROUND OF THE INVENTION

1. Field of the Invention

The present invention concerns a method and a magnetic resonance system for implementing a magnetic resonance-assisted biopsy examination.

2. Description of the Prior Art

For a biopsy examination it can be advantageous to exactly localize the position of an extraction region (for example a region with a tumor tissue) of a patient again (after an initial examination) just before a sample extraction and/or tissue extraction. Such a localization measurement can be done using a magnetic resonance system.

For the localization measurement the patient is positioned on a patient bed and a monitoring acquisition is implemented using the magnetic resonance system. An evaluation of measurement data subsequently takes place in which exact coordinates for the biopsy are determined. However, this evaluation takes place by means of a computer console that is placed outside of an examination space for the magnetic resonance measurement in order to prevent an unwanted disruption of the magnetic resonance measurement by the computer console. To determine the biopsy coordinates, the operator must leave the examination space in which the magnetic resonance examination occurs. Only after the evaluation can the operator return to the examination space with the determined biopsy coordinates and there initiate the biopsy examination, for example by positioning a biopsy needle using the calculated coordinates. This method has the disadvantage that the operator must physically take the biopsy coordinates into the examination space in some memorialized form, since an automatic data transfer from the computer console into the examination space is not provided.

This known procedure additionally has the disadvantage of being very time-consuming and requiring a long residence time for the evaluation, primarily between the monitoring measurement and the actual biopsy. Movements of the patient can occur during this time, which movements can lead to inaccuracies in the determination of the biopsy coordinates. An additional disadvantage of this method is that it is only rarely implemented, due to its very large time requirement, and therefore it is not among the regular routine tasks of the operator. This can lead to a high probability of error in the implementation of individual method steps of this method.

### SUMMARY OF THE INVENTION

An object of the present invention is to provide a magnetic resonance biopsy method with a short implementation time and an error-free implementation.

The invention proceeds from a magnetic resonance biopsy method for a biopsy examination, supported by at least one magnetic resonance measurement, made with a magnetic resonance measurement unit, a biopsy unit and a computer console, wherein the computer console is arranged separately from the magnetic resonance measurement unit that includes the following method steps:
a preparation step to position a patient on a patient bed,
a measurement step to acquire a magnetic resonance measurement of a region of the patient that is relevant to the biopsy examination,
a planning step to plan a sample extraction,
a biopsy step for sample extraction from the region of the patient that is relevant to the biopsy examination.

In accordance with the invention, the operator is guided through the preparation step and/or the measurement step and/or the planning step and/or the biopsy step in a computer-controlled manner. As used herein, a computer console means a unit that is designed to evaluate and/or control and/or regulate etc., this unit having a processor and/or additional units and/or components. The computer console and the magnetic resonance measurement unit are located in different rooms in order to obtain an undistorted magnetic resonance measurement and/or an undistorted data evaluation, but are advantageously connected with one another to exchange information and/or parameters. An advantageous reduction of unwanted operator errors and, additionally, a reliable guidance of an operator—for example medical personnel—through a complex method workflow of the magnetic resonance biopsy method can be achieved according to the invention. Furthermore, a time-saving implementation of the magnetic resonance biopsy method can take place by—for example—precise direction instructions that lead to a fast method workflow. A guidance of the individual method steps of the magnetic resonance biopsy method can take place by means of an at least partial graphical presentation of parameters and/or instructions for the operator. The at least partial graphical presentation of the instructions takes place automatically at least in part by means of a display unit (for example a monitor and/or a display) of the magnetic resonance measurement unit. Control of the computer-controlled guidance advantageously takes place via the guidance unit that is formed by (at) the computer console. The operator is guided in a computer-controlled manner through the preparation step and/or the biopsy step.

A workflow sequence of the individual method steps and/or individual sub-steps of a method step can be predetermined, so an advantageous time savings can be achieved by avoiding an incorrect operation and/or an incorrect workflow sequence of the individual method steps and/or sub-steps. Patient comfort can additionally be increased because the treatment time for the magnetic resonance biopsy method can be minimized. A guidance adapted to a work speed of the operator can advantageously be achieved by requiring confirmation information to be input as an entry after an implementation of at least one method step and/or a sub-step of a method step. An unintended skipping of a method step and/or sub-step is additionally ensured by requiring the operator to confirm the execution of the method step and/or of the sub-step again. For this purpose, the magnetic resonance measurement unit advantageously has an input unit via which the confirmation information can be entered.

In an embodiment a fault-detection procedure can be executed to detect faulty operation in at least one method step and/or a sub-step of a method step. An immediate correction of the faulty operation can thus take place. Untrained operators can additionally also be directed safely through the magnetic resonance biopsy method.

In an embodiment of the invention at least one biopsy parameter is generated as an output. The output of the at least one biopsy parameter advantageously takes place directly at the magnetic resonance measurement unit by means of the display unit. An advantageous time savings can be achieved by the extraction taking place promptly relative to the preparation steps. A reconstruction of the biopsy parameters advantageously takes place at least partially automatically by means of the computer console. The biopsy parameter can be a biopsy coordinate and/or a penetration depth and/or a penetration angle etc., for example.

Furthermore, the invention encompasses from a magnetic resonance system with a computer console, a magnetic resonance measurement unit and a biopsy unit, wherein the magnetic resonance system is provided for an implementation of a magnetic resonance biopsy method for a biopsy examination—assisted by at least one magnetic resonance measurement—with the following method steps:
a preparation step for positioning a patient on a patient bed,
a measurement step to acquire a magnetic resonance measurement of a region of the patient that is relevant to the biopsy examination,
a planning step to plan a sample extraction and
a biopsy step for a sample extraction from the region of the patient that is relevant to the biopsy examination.

In accordance with the invention, the magnetic resonance system has a guidance unit that is designed for guidance through the preparation step and/or the measurement step and/or the planning step and/or the biopsy step. The guidance unit is advantageously formed by the computer console. The computer console is fashioned separately from the magnetic resonance measurement unit and, to avoid an interference with a magnetic resonance examination, is located outside of a space provided by the examination room for the magnetic resonance examination. An advantageous reduction of unwanted operating errors and additionally a secure guidance of the operator through a complex method workflow of the magnetic resonance biopsy method can be achieved. Furthermore, a time-saving implementation of the magnetic resonance biopsy method can be achieved by (for example) precise guidance instructions leading to a fast method workflow. For this purpose, the magnetic resonance measurement unit advantageously has a display unit, for example a monitor and/or a display so that the guidance of the individual method steps of the magnetic resonance biopsy method can hereby take place by means of an at least partially graphical presentation of instructions. The operator is directed in a computer-controlled manner through the preparation step and/or the biopsy step.

Furthermore, the magnetic resonance system can have an error-detection unit to detect operating errors, so incorrect measurements can advantageously be prevented. An immediate correction of the incorrect operation can additionally be achieved, in particular if this is indicated to the operator immediately after a detection of the incorrect operation.

The biopsy device is at least partially magnetic resonance-compatible and is advantageously fashioned so as to be completely magnetic resonance compatible so that an unwanted interference with the magnetic resonance measurement can be prevented by the biopsy device. A magnetic resonance-compatible design means that the biopsy device is formed from non-magnetizable material.

In a further embodiment, the magnetic resonance system additionally has a contrast agent unit that is fashioned at least in part to be magnetic resonance-compatible, to allow administration of a contrast agent during the magnetic resonance biopsy method while avoiding unwanted interference.

### BRIEF DESCRIPTION OF THE DRAWINGS

FIG. 1 schematically illustrates a magnetic resonance system according to the invention.

FIG. 2 schematically illustrates a biopsy unit of the magnetic resonance system.

FIG. 3 is a flow chart of the basic steps of a method according to the invention.

FIG. 4 is an example of a first screen display for guidance of a biopsy procedure in accordance with the invention.

FIG. 5 is an example of a second screen display for guidance of a biopsy procedure in accordance with the invention.

FIG. 6 is an example of a third screen display for guidance of a biopsy procedure in accordance with the invention.

### DESCRIPTION OF THE PREFERRED EMBODIMENTS

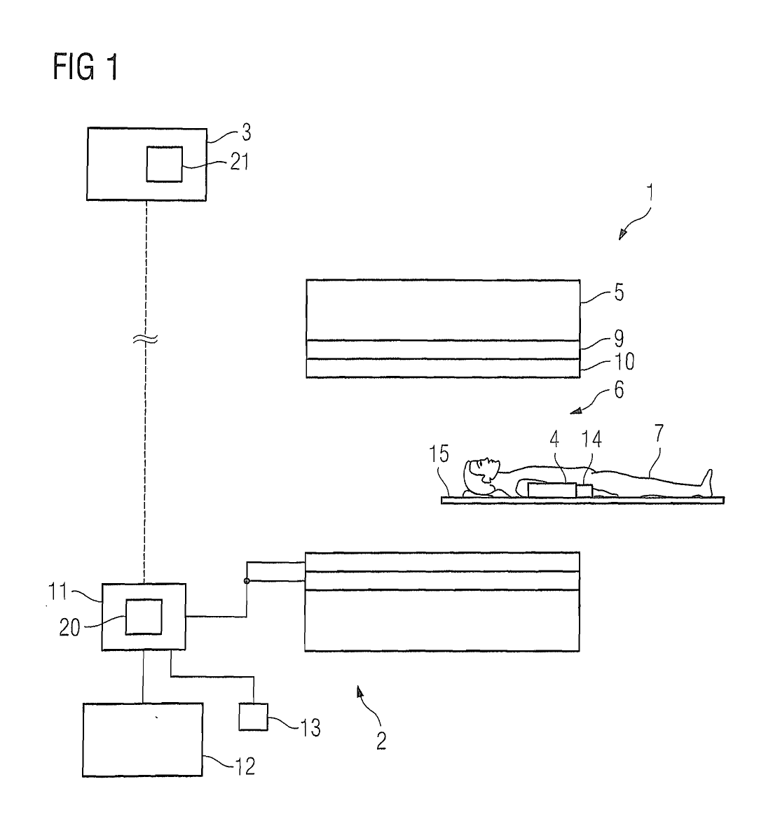

A magnetic resonance system 1 according to the invention, with a magnetic resonance measurement unit 2, a computer console 3 and a biopsy unit 4, is shown in FIG. 1. The magnetic resonance measurement unit 2 has a magnet 5 to generate a basic magnetic field. The magnetic resonance measurement unit 2 additionally has a cylindrical acquisition region 6 to acquire a patient 7. The patient 7 can be slid into the acquisition region 6 by means of a patient bed 8. The magnetic resonance measurement unit 2 furthermore has a gradient unit 9 to generate magnetic field gradients that are used for an imaging and a spatial coding. Furthermore, the magnetic resonance measurement unit 2 has a radio-frequency coil unit 10 to excite a polarization that arises in the basic magnetic field generated by the magnet 5.

To control the magnet 2, the gradient unit 9 and the radio-frequency coil unit 10, the magnetic resonance measurement unit 2 has a control unit 11. Control information (for example imaging parameters) and reconstructed magnetic resonance images can be displayed on a display unit 12 of the magnetic resonance measurement unit 2. The magnetic resonance measurement unit 2 additionally has an input unit 13 by means of which information and/or parameters can be input by an operator during a measurement process.

The magnetic resonance measurement unit 2 schematically shown in FIG. 1 can naturally comprise additional components that a magnetic resonance measurement unit 2 conventionally possesses. The general manner of operation of a magnetic resonance measurement unit 2 is known to those skilled in the art, such that a more detailed description of the general components is not necessary herein.

The magnetic resonance system 1 additionally has a contrast agent unit 14 by means of which a contrast agent for a magnetic resonance measurement can be administered. For this the contrast agent unit 14 is fashioned so as to be magnetic resonance-compatible.

The computer console 3 of the magnetic resonance system 1 is fashioned separate from the magnetic resonance measurement unit 2. The computer console 3 is additionally arranged to outside of an examination space provided for the magnetic resonance measurement or the magnetic resonance unit 2 to avoid unwanted interferences with and/or impairments of the magnetic resonance measurement. A data transfer between the control unit 11 of the magnetic resonance measurement unit 2 and the computer console 3 (that, for example, are connected with one another by means of a data line) takes place via a respective data interface A data evaluation of magnetic resonance measurements takes place by means of the computer console. The computer console 3 has a guidance unit 21, an input unit (not shown in detail) and a display unit (not shown in detail).

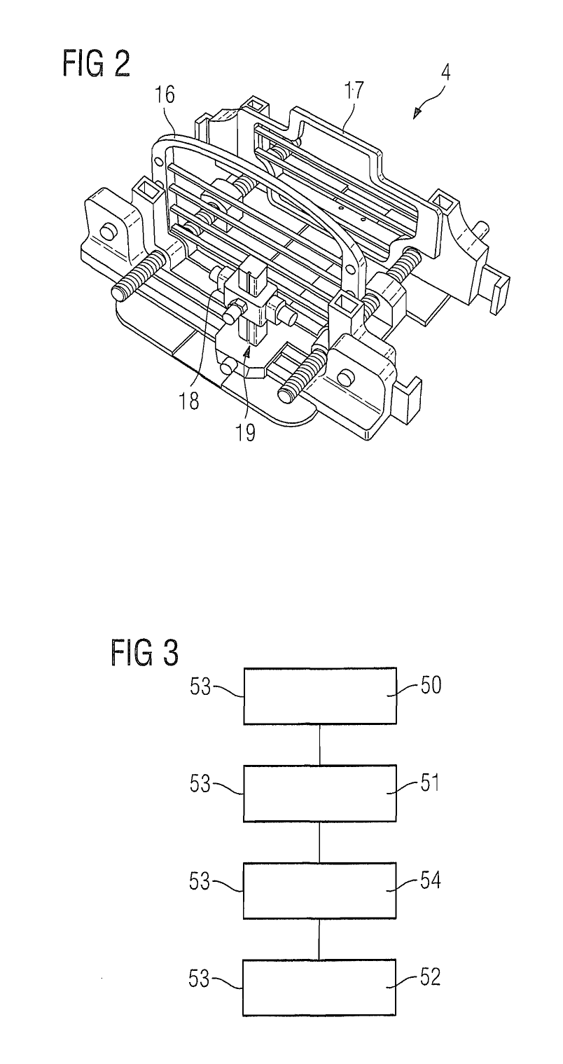

The biopsy unit 4 is likewise fashioned to be magnetic resonance-compatible and is shown in detail in FIG. 2. The biopsy unit 3 is formed by a breast biopsy unit and has compression elements 16, 17 that are provided for a compression of the region of the patient that is relevant to the biopsy examination (for example a breast region) so that this is essentially borne for a sample extraction such that it cannot slip. The biopsy unit 4 additionally has a target unit 18 and a marking element 19. The marker element 19 is provided to mark a reference position during the magnetic resonance measurement. The target unit 18 is provided to adjust a needle position of a biopsy needle of the biopsy unit 4 for the biopsy.

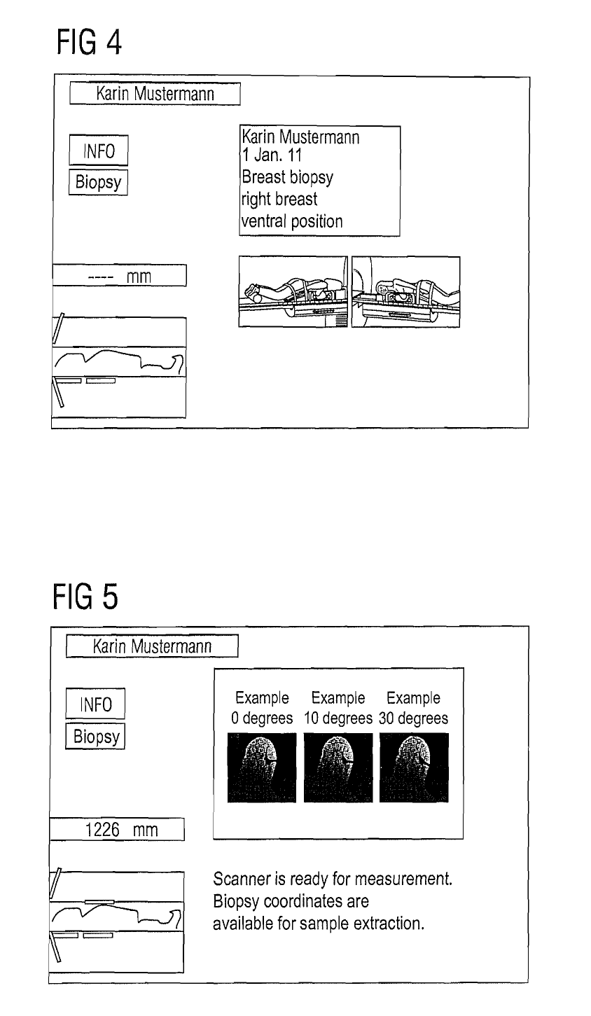

A method workflow of the magnetic resonance biopsy method according to the invention is shown in FIG. 3. After an operator has selected the magnetic resonance biopsy method via the display unit and the input unit of the computer console 3, the magnetic resonance biopsy method is automatically started by the guidance unit 21. In the magnetic resonance biopsy method the operator is guided by the individual method steps 53, wherein the guidance by the guidance unit 21 is controlled automatically. For this purpose, the guidance unit 21 generates guidance parameters and guidance instructions that are transferred into the examination space by means of the data interfaces. The instructions and/or indications generated by the guidance unit 21 in the individual method steps 53 are communicated to the operator via the display unit 12 of the magnetic resonance measurement unit 2, as this can be learned in an example from FIG. 4 through 6. As an alternative to this it is conceivable that a selection of the magnetic resonance biopsy method at the magnetic resonance measurement unit 2 can be made by an operator by means of the display unit 12 and input unit 13.

The magnetic resonance biopsy method includes a preparation step 50, a measurement step 51, a planning step 54 and a biopsy step 52. The preparation step 50 comprises multiple sub-steps, for example a placement of a local coil on the patient bed 8, a positioning of support aids for the patient 7, an attachment of mounting devices for the local coil etc. In addition to this, the patient 7 is likewise prepared for the examination in the preparation step 50, for example in that a region that is relevant to the biopsy examination is specifically borne on the patient bed 8. For example, a breast to be examined can be stabilized by means of compression plates (FIG. 4). With regard to a biopsy examination, the preparation step 50 comprises multiple sub-steps, for example a placement of the biopsy unit 4 on the patient bed 8, a monitoring and use of the marker element 19 etc. The operator is directed via these sub-steps, wherein the guidance takes place automatically, in a computer-controlled manner by means of the guidance unit 21. A graphical presentation of instructions and/or parameters to be set hereby takes place at least in part via the display unit 12. Control of the display unit 12 takes place via the guidance unit 21. A contrast agent can additionally be injected in the preparation step, wherein the contrast agent administration takes place via the contrast agent unit 14.

After the preparation step 50 the measurement step 51 takes place to acquire the magnetic resonance measurement of a region of the patient 7 that is relevant to the biopsy examination, for example a breast region. In the measurement step 51 a precise position of the region relevant to the biopsy examination—for example a lesion—is detected. The planning step 54 follows the measurement step 51. In the planning step 54 a planning and/or preparation of the biopsy examination takes place that comprises multiple sub-steps, for example a detection of the reference marking within the magnetic resonance acquisition, a calculation of biopsy coordinates etc. The measurement step 51 and the planning step 54 for the biopsy examination are implemented by the operator at the computer console 3.

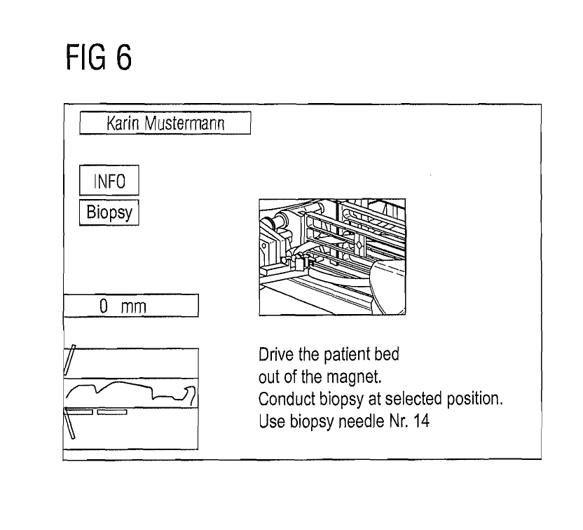

The biopsy step 52 follows the planning step 54. The sample extraction takes place in this biopsy step 52 (FIGS. 5 and 6). For the sample extraction the patient must be driven out of the acquisition region 6 of the magnetic resonance measurement unit 2 and, in addition to this, the biopsy coordinates output by the magnetic resonance measurement unit 2 must still be set at the target unit 18. The biopsy coordinates are shown directly above the display unit 12 of the magnetic resonance measurement unit for the operator, such that the operator can set these coordinates directly. Multiple different biopsy coordinates from different penetration directions can hereby be displayed to the operator (FIGS. 5 and 6). If all preparation steps have occurred, the operator receives the request and/or the instruction to conduct the sample extraction, wherein the size of a needle (cannula) and/or additional parameters and/or information provided for this is/are indicated to the operator.

A workflow sequence of the individual method steps 53 is provided by the guidance unit 21 in that the individual sub-steps of a method step 53 are displayed in a predetermined order to the operator via the display unit 12. Given complex sub-steps, a procedure and/or order of individual sub-steps can additionally be predetermined and communicated to the operator via the display unit 12 so that a correct and time-saving workflow of the individual method steps 53 and/or sub-steps of the method steps 53 is achieved. For this the guidance unit 21 has a workflow program for the magnetic resonance biopsy method that is stored in a memory unit (not shown in detail).

After an implementation and/or execution of the instructions of a sub-step and/or method step 53 (shown on the display element 12), via the input element 13 the operator can confirm their implementation and/or execution so that the next method step 53 in the workflow plan and/or the order can be displayed to the operator for correct execution. The input element 13 can hereby be formed by a button element and/or be fashioned in one piece with the display element 12, for example a design as a touchscreen.

Furthermore, the magnetic resonance measurement unit 2 has an error detection unit 20 by means of which an incorrect operation in at least one of the method steps 53 and/or in at least one sub-step of a method step 53 is detected. For example, in the preparation step 50 an incorrect positioning of the patient 7 for a selected examination region and/or an incorrect positioning of an arrangement of the local coil etc. can be detected. After a detection of the incorrect operation an output of an instruction to the operator for the incorrect operation takes place. Via the display unit 12 the operator is additionally newly requested and/or instructed to execute this method step 53 and/or this sub-step again.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for conducting a magnetic resonance-assisted biopsy, comprising the steps of:
in a preparation step, positioning a patient on a patient bed of a magnetic resonance data acquisition unit of a magnetic resonance imaging system;
in a measurement step, operating said magnetic resonance data acquisition unit to acquire magnetic resonance data representing an image of a region of the patient at which a biopsy examination is to be implemented;
in a planning step, planning extraction of a sample from said region of said patient;
in a biopsy step, extracting said sample from said region of the patient; and
at a computer located remotely from said data acquisition unit, guiding an operator, by computer control, through at least one of said preparation step, said measurement step, said planning step and said biopsy step.

2. A method as claimed in claim 1 comprising guiding said operator at said computer by displaying, at a display screen of said computer, a presentation of information selected from the group consisting of parameters and instructions in at least partially graphical form, for said at least one of said preparation step, said measurement step, said planning step and said biopsy step.

3. A method as claimed in claim 1 comprising executing said biopsy examination in a predetermined workflow sequence selected from the group consisting of a predetermined sequence of said preparation step, said measurement step, said planning step and said biopsy step, and a predetermined sequence of sub-steps in at least one of said preparation step, said measurement step, said planning step and said biopsy step.

4. A method as claimed in claim 3 comprising, at said computer, requiring entry of a confirmation input upon completing a step or a sub-step in said workflow sequence before permitting said biopsy examination to continue with a next step or sub-step in said workflow sequence.

5. A method as claimed in claim 3 comprising, at said computer, detecting an attempt to implement a step or sub-step in an out-of-sequence manner in said workflow sequence.

6. A method as claimed in claim 1 comprising emitting at least one biopsy parameter as an output from said computer.

7. A method as claimed in claim 1 comprising locating said magnetic resonance data acquisition unit in an examination room, and locating said computer outside of said examination room.

8. A magnetic resonance imaging system for implementing a magnetic resonance-assisted biopsy examination, comprising:
a magnetic resonance data acquisition unit having a patient bed;
said data acquisition unit being operable to, in a preparation step, positioning a patient on the patient bed, and in a measurement step, acquire magnetic resonance data representing an image of a region of the patient at which a biopsy examination is to be implemented, and in a planning step, plan extraction of a sample from said region of said patient; and in a biopsy step, extract said sample from said region of the patient; and
a computer located remotely from said data acquisition unit, configured to guide an operator, by computer control, through at least one of said preparation step, said measurement step, said planning step and said biopsy step.

9. A magnetic resonance system as claimed in claim 8 wherein said computer is configured to guide said operator at said computer by displaying, at a display screen of said computer, a presentation of information selected from the group consisting of parameters and instructions in at least partially graphical form, for said at least one of said preparation step, said measurement step, said planning step and said biopsy step.

10. A magnetic resonance system as claimed in claim 8 wherein said computer is configured to guide execution of said biopsy examination in a predetermined workflow sequence selected from the group consisting of a predetermined sequence of said preparation step, said measurement step, said planning step and said biopsy step, and a predetermined sequence of sub-steps in at least one of said preparation step, said measurement step, said planning step and said biopsy step.

11. A magnetic resonance system as claimed in claim 10 wherein said computer is configured to require entry of a confirmation input upon completing a step or a sub-step in said workflow sequence before permitting said biopsy examination to continue with a next step or sub-step in said workflow sequence.

12. A magnetic resonance system as claimed in claim 10 wherein said computer is configured to detect an attempt to implement a step or sub-step in an out-of-sequence mariner in said workflow sequence.

13. A magnetic resonance system as claimed in claim 8 wherein said computer is configured to emit at least one biopsy parameter as an output from said computer.

14. A magnetic resonance system as claimed in claim 8 wherein said magnetic resonance data acquisition unit is located in an examination room, and said computer is located outside of said examination room.
